(19) United States
(12) Patent Application Publication
(10) Pub. No.: US 2010/0210905 A1
(43) Pub. Date: Aug. 19, 2010

(54) ENDOSCOPE

(75) Inventors: Akio Takeuchi, Hino-shi (JP); Yoshiaki Ito, Fuchu-shi (JP)

Correspondence Address: Frishauf, Holtz Goodman & Chick, P.C., 220 Fifth Avenue - 16th Floor, New York, NY, 10001-7708, US

(73) Assignee: Olympus Corporation, Tokyo (JP)

(21) Appl. No.: 12/769,968

(22) Filed: Apr. 29, 2010

Related U.S. Application Data
(63) Continuation of application No. PCT/JP2008/069473, filed on Oct. 27, 2008.

(30) Foreign Application Priority Data
Nov. 26, 2007 (JP) 2007-304872

(51) Int. Cl. A61B 1/04

(52) U.S. Cl. 600/110; 600/112

(57) ABSTRACT

An endoscope includes an elongated insertion portion inserted into a lumen, an operation portion coupled with a proximal end portion of the insertion portion, and an imaging unit which is arranged in a distal end portion of the insertion portion and which is configured to acquire an endoscopic image. The endoscope includes an imaging unit attachment, portion which is provided at the distal end portion of the insertion portion and detachably holds the imaging unit, an imaging unit insertion/removal portion which is provided in the operation portion and enables insertion/removal of the imaging unit with respect to the insertion portion, and an operating mechanism which includes a distal end portion fixed to the imaging unit and a proximal end portion extended to a imaging unit insertion/removal portion side, and which is configured to perform an attachment/detachment operation of the imaging unit on the imaging unit insertion/removal portion side.

### CROSS REFERENCE TO RELATED APPLICATIONS

This is a Continuation Application of PCT Application No. PCT/JP2008/069473, filed Oct. 27, 2008, which was published under PCT Article 21(2) in Japanese.

This application is based upon and claims the benefit of priority from prior Japanese Patent Application No. 2007-304872, filed Nov. 26, 2007, the entire contents of which are incorporated herein by reference.

### BACKGROUND OF THE INVENTION

1. Field of the Invention

The present invention relates to an endoscope including an imaging unit which acquires an endoscopic image.

2. Description of the Related Art

In general, it is often the case that an imaging unit which acquires an endoscopic image is incorporated in a distal end portion of an insertion portion of an endoscope. Endoscopes each of which has a configuration that the image pickup portion is separated as an imaging unit from a main boy of an endoscope have been conventionally disclosed.

For example, Published Japanese Patent No. 3356355 (Patent Document 1) discloses the following configuration. Here, engaging mechanism which is configured to detachably hold a distal end portion of an insertion portion and an imaging unit are provided. As a result, a distal end side portion of the imaging unit can be attached to/detached from the insertion portion.

JP-A 07-275197 (KOKAI) (Patent Document 2) discloses an endoscope including the following configuration. Here, an accommodation portion of an image pickup portion, which is opened to a distal end side of an insertion portion of an electronic endoscope main body, is provided. The image pickup portion can be put into or removed from the accommodation portion from a distal end face of the electronic endoscope main body which is opened. A collection wire is connected to the image pickup portion. Further, at the time of acquiring an endoscopic image, the image pickup portion can be separated from the accommodation portion in a state that the image pickup portion protrudes to the outside of the accommodation portion. Furthermore, the separated image pickup portion can be retracted into the accommodation portion toward the endoscope main body side by pulling the collection wire.

### BRIEF SUMMARY OF THE INVENTION

According to one aspect of the invention, there is provided An endoscope comprising: an elongated insertion portion which is inserted into a lumen; an operation portion which is coupled with a proximal end portion of the insertion portion; an imaging unit which is arranged in a distal end portion of the insertion portion and which is configured to acquire an endoscopic image; an imaging unit attachment portion which is provided at the distal end portion of the insertion portion and detachably holds the imaging unit; an imaging unit insertion/removal portion which is provided in the operation portion and enables insertion/removal of the imaging unit with respect to the insertion portion; and an operating mechanism which includes a distal end portion fixed to the imaging unit and a proximal end portion extended to a imaging unit insertion/removal portion side, and which is configured to perform an attachment/detachment operation of the imaging unit with respect to the imaging unit attachment portion on the imaging unit insertion/removal portion side.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a perspective view of an endoscope according to a first embodiment of the present invention;

FIG. 2 is a plan view of a distal end face of the endoscope according to the first embodiment;

FIG. 3 is a cross-sectional view taken along line in FIG. 2;

FIG. 4 is a perspective view of an imaging unit of the endoscope according to the first embodiment;

FIG. 5 is a vertical sectional view of the imaging unit of the endoscope according to the first embodiment;

FIG. 6 is a vertical sectional view showing an imaging unit attachment portion of the endoscope according to the first embodiment;

FIG. 7 is a perspective view showing an imaging unit of an endoscope according to a second embodiment of the present invention;

FIG. 8 is a vertical sectional view showing the imaging unit of the endoscope according to the second embodiment;

FIG. 9 is a perspective view showing an imaging unit of an endoscope according to a third embodiment of the present invention;

FIG. 10 is a vertical sectional view showing the imaging unit of the endoscope according to the third embodiment;

FIG. 11 is a perspective view of a primary part showing a state before an imaging unit is assembled to an imaging unit attachment portion of an endoscope according to a fourth embodiment of the present invention;

FIG. 12 is a vertical sectional view of a primary part showing a state that the imaging unit is assembled to the imaging unit attachment portion of the endoscope according to the fourth embodiment;

FIG. 13A is a plan view showing a first modification of the imaging unit according to the fourth embodiment;

FIG. 13B is a plan view showing a second modification of the imaging unit according to the fourth embodiment;

FIG. 130 is a plan view showing a third modification of the imaging unit according to the fourth embodiment;

FIG. 14 is a vertical sectional view of a primary part showing a state before an imaging unit is assembled to an imaging unit attachment portion of an endoscope according to a fifth embodiment of the present invention;

FIG. 15 is a vertical sectional view of a primary part showing a state that the imaging unit is assembled to the imaging unit attachment, portion of the endoscope according to the fifth embodiment;

FIG. 16 is a vertical sectional view of a primary part showing a state that the imaging unit is unlocked from the imaging unit attachment portion of the endoscope according to the fifth embodiment; and

FIG. 17 is a vertical sectional view of a primary part showing a state that the imaging unit is removed from the imaging unit attachment portion of the endoscope according to the fifth embodiment.

### DETAILED DESCRIPTION OF THE INVENTION

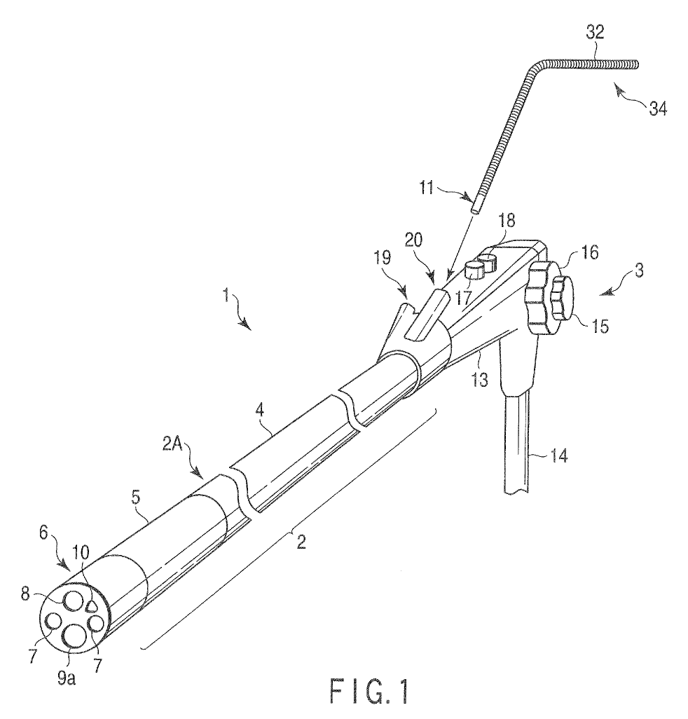

A first embodiment according to the present invention will now be described hereinafter with reference to FIGS. 1 to 6. FIG. 1 shows an example of an endoscope 1 according to this embodiment. This endoscope 1 has an elongated insertion portion 2 which is inserted into a body and an operation portion 3 coupled with a proximal end portion of this insertion portion 2. A main body 2A of the insertion portion 2 has an elongated flexible tube portion 4, a bending portion 5 whose proximal end portion is coupled with a distal end of this flexible tube portion 4, and a distal end hard portion 6 whose proximal end portion is coupled with a distal end of this binding portion 5.

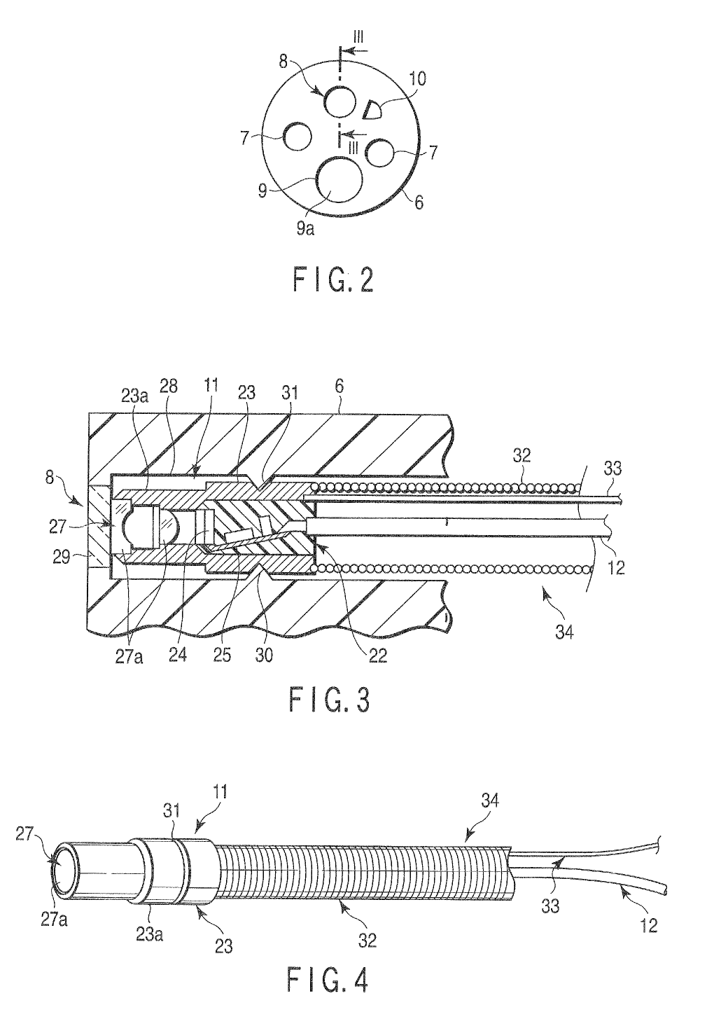

In a distal end face of the distal end hard portion 6, two illumination window portions 7 of an illumination optical system, an observation window portion 8 of an observation optical system, a distal end opening portion 9a of a surgical instrument insertion channel 9, an air supply/water supply nozzle 10, and others are arranged as shown in FIG. 2.

In the distal end hard portion 6, a distal end portion of a non-illustrated light guide fiber is fixed behind each illumination window portion 7. Further, an imaging unit 11 which acquires an endoscopic image is arranged behind the observation window portion 8 as depicted in FIG. 3. Furthermore, a distal end portion of the surgical instrument insertion channel 9, a distal end portion of a non-illustrated air supply/water supply tube connected with the air supply/water supply nozzle 10, and others are fixed in the distal end hard portion 6.

The light guide fiber, a cable 12 such as an imaging signal line of the imaging unit 11, the surgical instrument insertion channel 9, the air supply tube or a water supply tube connected with the air supply/water supply nozzle 10 and others pass through the flexible tube portion 4 from the inside of the bending portion 5 to be extended to a proximal end side of the flexible tube portion 4.

Moreover, the operation portion 3 is coupled with the proximal end portion of the flexible tube portion 4. A grip portion 13 gripped by an operator is arranged in the operation portion 3. A proximal end portion of a universal cord 14 is coupled with a terminal portion of the grip portion 13. A connector portion connected with a non-illustrated light source device or video processor is coupled with a distal end portion of this universal cord 14.

Additionally, in the operation portion 3, a vertical bending operation knob 15 and a lateral bending operation knob 16 which operate the bending portion 5 to bend, a suction button 17, an air supply/water supply button 18, various kinds of switches for endoscopy, a surgical instrument insertion portion 19, and an imaging unit insertion/removal portion 20 are provided. In the surgical instrument insertion portion 19, a surgical instrument insertion opening coupled with the proximal end portion of the surgical instrument insertion channel 9 arranged in the insertion portion 2 is provided. Further, a non-illustrated endoscope surgical instrument is inserted into the surgical instrument insertion channel 9 from the surgical instrument insertion opening of the surgical instrument insertion portion 19 to be pushed to the distal end hard portion 6. Thereafter, the endoscope surgical instrument is made to protrude to the outside from the distal end opening portion 9a of the surgical instrument insertion channel 9.

As shown in FIG. 3, the imaging unit 11 in the distal end hard portion 6 has an imaging module portion 22 and a unit case 23 formed of, e.g., metal. The unit case 23 is provided on an outer periphery of the imaging module portion 22.

The imaging module portion 22 has an imaging element 24 such as a CCD and its associated electronic components, e.g., a connection circuit board 25 and others. A distal end portion of the cable 12, e.g., the imaging signal line is connected with the connection circuit board 25. The imaging element 24 and the connection circuit board 25 are molded by using a resin material. As a result, the imaging module portion 22 is integrally formed by using the resin material.

The unit case 23 has a cylindrical case main body 23a. A lens unit 27 is arranged at a distal end portion of a case main body 23a. The lens unit 27 includes a plurality of lenses 27a arranged in parallel and forming an observation optical system. The imaging module portion 22 is arranged to a rear side of the lens unit 27, and, at a rear end portion of the case main body 23a. Further, light, entering the observation window portion 8 is subjected to image formation on an imaging plane of the imaging element 24 of the lens unit 27, thereby acquiring an endoscopic image. At this time, photoelectric conversion is performed in the imaging element 24, and an output signal of image data of the endoscopic image acquired by the imaging element 24 is transmitted to the processor side portion such as a non-illustrated camera control unit through the imaging signal cable 12.

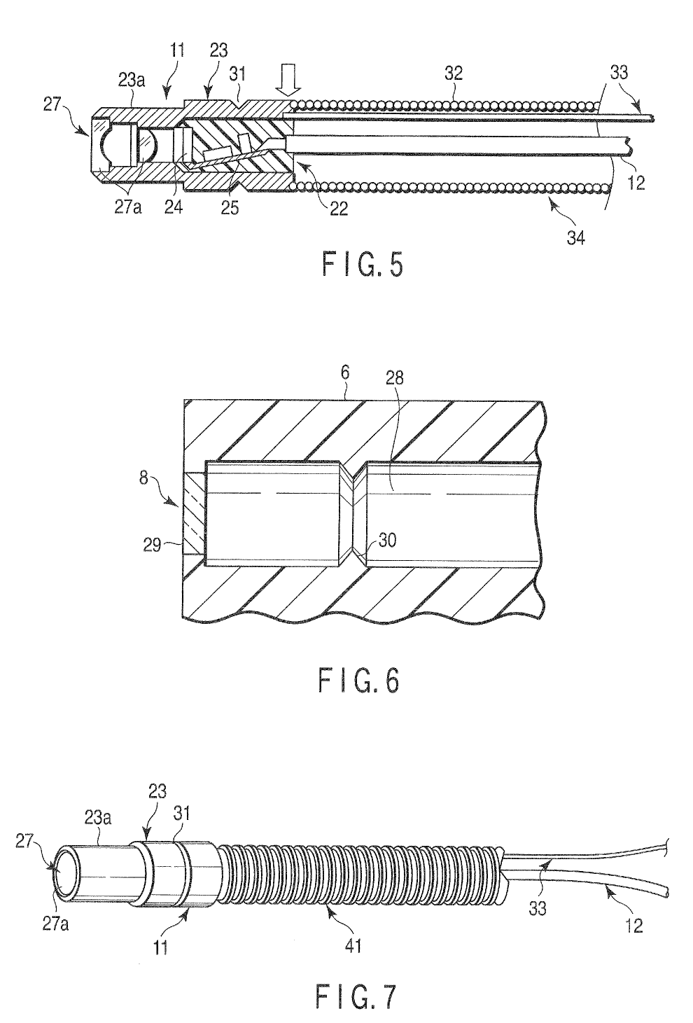

Meanwhile, an imaging unit attachment hole portion (an imaging unit attachment portion) 28 depicted in FIG. 6 is formed in the distal end hard portion 6 of the main body 2A of the insertion portion 2. A distal end opening portion of this imaging unit attachment hole portion 28 is closed by a transparent cover glass 29 fixed to the observation window portion 8.

As shown in FIG. 6, a ring-shaped protruding portion 30 is provided to protrude on an inner peripheral surface of the imaging unit attachment hole portion 28. The protruding portion 30 is a part that positions an axial fixing position of the imaging unit 11. A ring groove 31 is provided on an outer peripheral surface of the unit case 23 of the imaging unit 11 along the circumferential direction. This ring groove 31 is formed to have a substantially-V-shaped cross-sectional shape. The ring groove 31 is arranged at a position corresponding to the protruding portion 30 of the imaging unit attachment hole portion 28. Further, when attaching the imaging unit 11 in the imaging unit attachment hole portion 28, a distal end portion of the imaging unit 11 is moved to an attaching position where a distal end portion of the imaging unit 11 abuts on a cover glass 29 as shown in FIG. 3. At this position, the ring-like protruding portion 30 of the imaging unit attachment hole portion 28 can be detachably engaged with the ring groove 31 of the imaging unit 11. As a result, a fixing position of the imaging unit 11 in the axial direction can be positioned.

Furthermore, a distal end portion of a closely wound coil for a push-in operation (a push-out force transmission member) 32 and a distal end portion of an operation wire for a pull-out operation (a tensile force transmission member) 33 are fixed at a rear end portion of the imaging unit 11 as shown in FIG. 5. Here, a distal end portion of the closely wound coil 32 is welded and fixed to an outer peripheral surface of the rear end portion of the case main body 23a of the unit case 23. Moreover, a distal end portion of the operation wire 33 is welded and fixed to an inner peripheral surface of the rear end portion of the case main body 23a of the unit case 23. Additionally, the operation wire 33 and the imaging signal cable 12 are inserted into the closely wound coil 32.

Further, the rear end portions of the closely wound coil 32, the operation wire 33 and the imaging signal cable 12 are respectively extended to the imaging unit insertion/removal portion 20 side and extended to the outer side of the imaging unit insertion/removal portion 20. Here, the closely wound coil 32 forms pushing mechanism configured to be able to perform a push-in operation without reducing a length in a push-in direction. The operation wire 33 constitutes pull-out force transmission mechanism configured to be able to transmit pull-out force without extending beyond a given fixed length in a pull-out direction. Furthermore, the closely wound coil 32 and the operation wire 33 form operating mechanism 34 configured to perform an attachment/detachment operation of the imaging unit 11 to/from the imaging unit attachment hole portion 28 on the imaging unit insertion/removal portion 20 side portion.

A function of the above configuration will now be described. In the endoscope 1 according to this embodiment, the imaging unit 11 is pushed in from an opening portion (an imaging unit insertion opening) of the imaging unit insertion/removal portion 20 at the rear end of the insertion portion 2 to be inserted into the insertion portion 2, at the time of an operation of attaching/detaching the imaging unit 11 to/from the imaging unit attachment hole portion 28 on the main body 2A side portion of the insertion unit 2 (at the time of attaching the imaging unit 11). At this time, when the closely wound coil 32 is pushed in from the outer side of the imaging unit insertion/removal portion 20 at the rear end of the insertion unit 2, the push-in force is transmitted to the metal unit case 23 that covers the outer peripheral surface of the imaging unit 11 via the closely wound coil 32.

Moreover, the imaging unit 11 is pushed to the distal end side of the insertion portion 2 to be inserted into the imaging unit attachment hole portion 28. On the last stage of this insertion operation, the ring-like protruding portion 30 of the imaging unit attachment hole portion 28 is detachably engaged with the V-shaped ring groove 31 of the imaging unit 11 as shown in FIG. 3. As a result, in a state that the fixing position of the imaging unit 11 in the axial direction is positioned at the attaching position where the distal end portion of the imaging unit 11 abuts on the cover glass 29, the imaging unit 11 is fixed to the imaging unit attachment hole portion 28.

Additionally, at the time of a separating operation of separating the imaging unit 11 from the main body 2A of the insertion portion 2, an operation of pulling the operation wire 33 from the outer side of the imaging unit insertion/removal portion 20 at the rear end of the insertion portion 2 is performed. In this case, the tensile force is transmitted to the metal unit case 23 that covers the outer peripheral surface of the imaging unit 11 via the operation wire 33. This pull-our operation releases the engagement achieved between the ring-like protruding portion 30 of the imaging unit attachment hole portion 28 and the V-shaped ring groove 31 of the imaging unit 11, whereby the imaging unit 11 is removed from the imaging unit attachment hole portion 28.

Thus, the above configuration demonstrates the following effect. That is, in the endoscope 1 according to this embodiment, the closely wound coil 32 is pushed in from the outer side of the imaging unit insertion/removal portion 20. As a result, a attaching operation of attaching the imaging unit 11 to the imaging unit attachment hole portion 28 of the main body 2A of the insertion portion 2 can be performed. Further, when the operation wire 33 is pulled from the outer side of the imaging unit insertion/removal portion 20, the imaging unit 11 can be removed and separated from the imaging unit attachment hole portion 28 of the main body 2A of the insertion portion 2. Therefore, since the imaging unit 11 can be attached to or detached from the imaging unit attachment hole portion 28 of the main body 2A of the insertion portion 2 by pushing or pulling the elongated closely wound coil 32 or the elongated operation wire 33 from the outer side of the imaging unit insertion/removal portion 20, an operator no longer needs an operation of directly grasping the small imaging unit 11. Therefore, the workability of en operation of attaching/detaching the imaging unit 11 to/from the imaging unit attachment hole portion 28 of the main body 2A of the insertion portion 2 can be improved as compared with the conventional examples, whereby the operation of attaching the imaging unit 11 to the imaging unit attachment hole portion 28 of the main body 2A of the insertion portion 2 or removing the imaging unit 11 from the imaging unit attachment hole portion 28 can be readily performed.

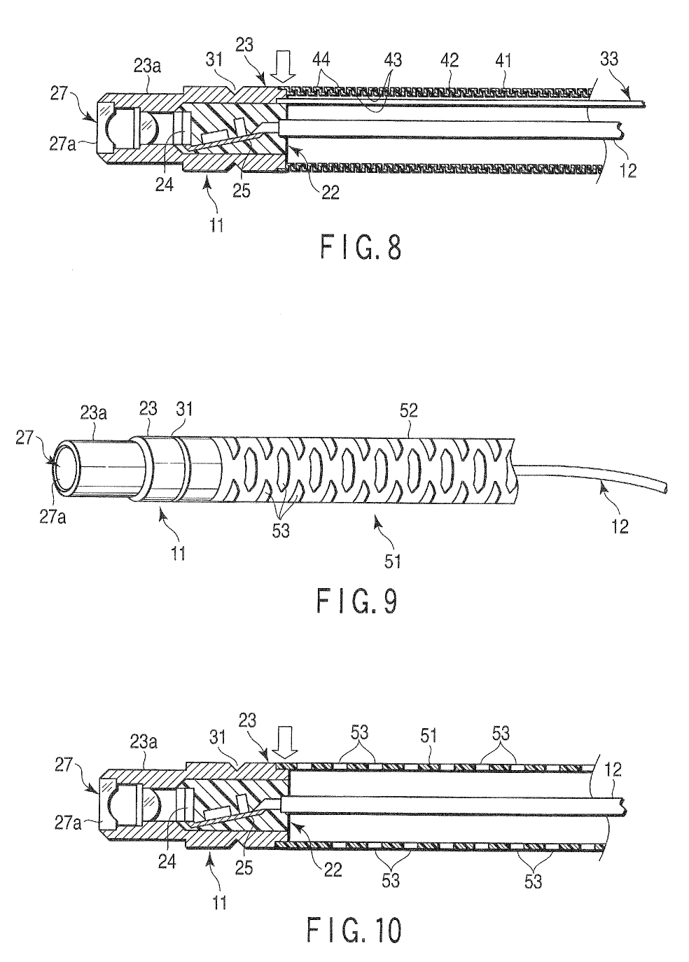

Furthermore, FIGS. 7 and 8 show a second embodiment according to the present invention. This embodiment is obtained by changing the configuration of the operating mechanism 34 configured to attach/detach the imaging unit 11 to/from the imaging unit attachment hole portion 28 of the main body 2A of the insertion portion 2 according to the first embodiment (see FIGS. 1 to 6) as follows.

That is, in this embodiment, a resin corrugated tube 41 is used in place of the closely wound coil 32 according to the first embodiment. The corrugated tube 41 is formed of a continuous tube body 42 having an irregular configuration as shown in FIG. 8. The continuous tube body 42 is obtained by alternately connecting a plurality of reduced-diameter portions 43 and a plurality of expanded-diameter portions 44 along the axial direction of the resin continuous tube body 42 having the flexibility. The corrugated tube 41 forms pushing mechanism configured to be able to perform a push-in operation without reducing a length in a push-in direction. Here, a distal end portion of the corrugated tube 41 is bonded and fixed to an outer peripheral surface of a rear end portion of a case main body 23a of a unit case 23. It is to be noted that the distal end portion of the corrugated tube 41 may be disposed and fixed to the outer peripheral surface of the rear end portion of the case main body 23a of the unit case 23 by fixation mechanism such as screwing using a non-illustrated fixation screw or thrust fixation using a securing ring.

Thus, in this embodiment, when the corrugated tube 41 is pushed in from an outer side of an imaging unit insertion/removal portion 20, a disposing operation of attaching the imaging unit 11 to an imaging unit attachment hole portion 28 of a main body 2A of an insertion portion 2 can be performed. Furthermore, when an operation wire 33 is pulled from the outer side of the imaging unit insertion/removal portion 20, an operation of removing and separating the imaging unit 11 from the imaging unit attachment hole portion 28 of the main body 2A of the insertion portion 2 can be performed. Therefore, since the imaging unit 11 can be attached to or detached from the imaging unit attachment hole portion 28 of the main body 2A of the insertion portion 2 by the operation of pushing/pulling the long corrugated tube 41 or the operation wire 33 from the outer side of the imaging unit insertion/removal portion 20, whereby an operator no longer needs an operation of directly grasping the small imaging unit 11. Accordingly, like the first embodiment, the workability of an operation of attaching/detaching the imaging unit 11 to/from the imaging unit attachment hole portion 28 of the main body 2A of the insertion portion 2 can be improved, thereby readily effecting an operation of incorporating the imaging unit 11 into the imaging unit attachment hole portion 28 of the main body 2A of the insertion portion 2 and an operation of removing the imaging unit 11 from the imaging unit attachment hole portion 28.

It is to be noted that the corrugated tube 41 according to this embodiment can be also used as pull-out force transmission mechanism configured to be able to transmit pull-out force without extending beyond a given fixed length in a tensile direction. Therefore, the corrugated tube 41 can be used as the single force transmission member which functions as both the push-out force transmission member and the tensile force transmission member. In this case, since the operation wire 33 utilized in the first embodiment is no longer necessary, an effect of further reducing the number of components can be obtained.

Moreover, FIGS. 9 and 10 show a third embodiment according to the present invention. This embodiment is obtained by changing the configuration of the operating mechanism 34 configured to attach/detach the imaging unit 11 to/from the imaging unit attachment hole portion 28 of the main body 2A of the insertion unit 2 according to the first embodiment (see FIGS. 1 to 6) as follows.

That is, in this embodiment, a perforated tube 51 is used as a force transmission member in place of the closely wound coil 32 according to the first embodiment. The perforated tube 51 is obtained by forming a plurality of hole portions 53 that adjust flexibility in a tube wall of a resin tube body 52. A number, a shape, a size and others of the hole portions 53 can be appropriately changed as required. Here, a distal end portion of the perforated tube 51 is bonded and fixed to an outer peripheral surface of a rear end portion of a case main body 23a of a unit case 23. It is to be noted that the distal end portion of the perforated tube 51 may be disposed and fixed to the outer peripheral surface of the rear end portion of the case main body 23a of the unit case 23 by fixation mechanism such as screwing using a non-illustrated fixing screw or thrust fixation using a securing ring.

The perforated tube 51 according to this embodiment constitutes push-in mechanism configured to be able to perform a push-in operation without reducing a length in a push-in direction, and it can be used as pull-out force transmission mechanism configured to be able to transmit pull-out force without extending beyond a given fixed length in a pull-out direction. Therefore, the perforated tube 51 can be used as the single force transmission member that can function as both a push-out force transmission member and a tensile force transmission member. Therefore, in this embodiment, the workability of an operation of attaching/detaching an imaging unit 11 to/from an imaging unit attachment hole portion 28 of a main body 2A of an insertion portion 2 can be improved as compared with conventional examples like the first embodiment, thereby obtaining an effect of easily performing an operation of incorporating the imaging unit 11 into the imaging unit attachment hole portion 28 of the main body 2A of the insertion portion 2 and removing the imaging unit 11 from the imaging unit attachment hole portion 28. Furthermore, since the operation wire 33 utilized in the first embodiment is no longer necessary, an effect of further reducing the number of components can be obtained.

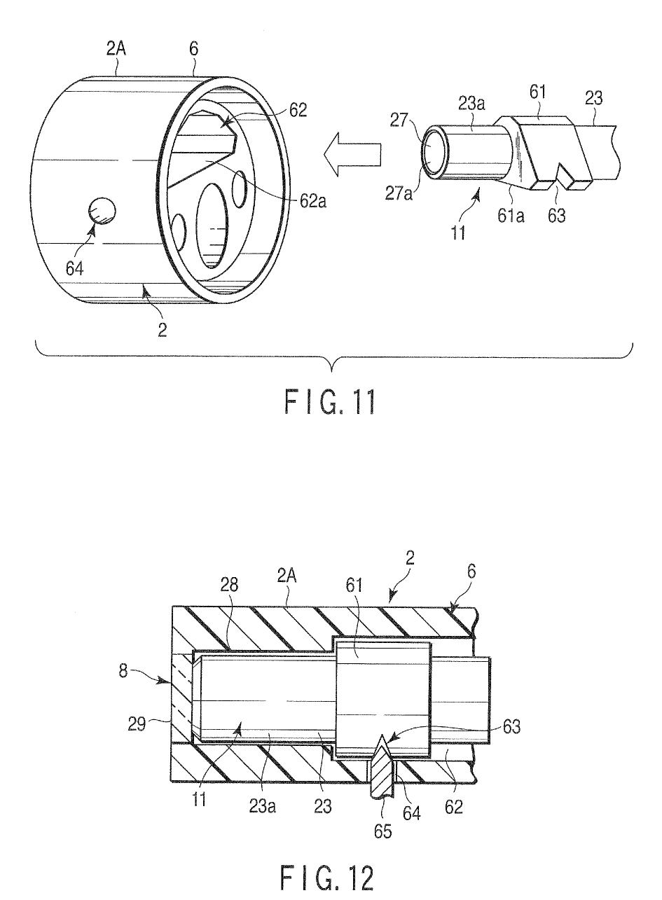

Moreover, FIGS. 11 and 12 show a fourth embodiment according to the present invention. This embodiment is obtained by changing the configuration of the engagement portion which engages the imaging unit attachment hole portion 28 of the main body 2A of the insertion portion 2 and the imaging unit 11 according to the first embodiment (see FIGS. 1 to 6) as follows.

That is, the configuration where the unit case 23 of the imaging unit 11 having the cylindrical case main body 23a is used as the engagement portion has been described in the first embodiment. In this embodiment, as shown in FIG. 11, an asymmetrical odd-shaped fitting portion 61 which partially protrudes is provided on an outer peripheral surface of a cylindrical case main body 23a along a periaxial direction of a central axis of an insertion portion 2. At least one flat surface 61a is provided to this odd-shaped fitting portion 61 in a circumferential direction of the outer peripheral surface of a unit case 23.

Additionally, an imaging unit attachment hole portion 28 of a main body 2A of an insertion portion 2 has a fitting hole portion 62 which fits to the odd-shaped fitting portion 61 of the unit case 23. The fitting hole portion 62 is formed into a shape associated with the odd-shaped fitting portion 61 of the unit case 23. Further, one flat surface 62a is likewise provided to the fitting hole portion 62 at a position associated with the flat surface 61a.

Further, in this embodiment, a V groove portion 63 is formed at an outer peripheral region (a region at a top portion formed to protrude) of the odd-shaped fitting portion 61. Here, in the main body 2A of the insertion portion 2, a wedge insertion hole 64 is formed in a circumferential wall portion of the fitting hole portion 62. This wedge insertion hole 64 is arranged at a position associated with the V groove portion 63 of the odd-shaped fitting portion 61 in a state that a fixing position of an imaging unit 11 in an axial direction is positioned at an attaching position where a distal end portion of the imaging unit 11 abuts on a cover glass 29 when the odd-shaped fitting portion 61 is inserted into the fitting hole portion 62.

Further, in a state that the fixing position of the imaging unit 11 in the axial direction is positioned at the attaching position where the distal end portion of the imaging unit 11 abuts on the cover glass 29 when the odd-shaped fitting portion 61 is inserted into the fitting hole portion 62, a distal end portion of a wedge 65 which is inserted from the wedge insertion hole 64 is inserted into and fixed to the V groove portion 63 as shown in FIG. 12. As a result, in the state that the fixing position of the imaging unit 11 in the axial direction is positioned at the attaching position where the distal end portion of the imaging unit 11 abuts on the cover glass 29, the movement of the imaging unit 11 in the axial direction is restricted with respect to the imaging unit attachment hole portion 28.

Thus, the above configuration demonstrates the following effect. That is, in this embodiment, when the imaging unit 11 is attached to the imaging unit attachment hole portion 28 of the main body 2A of the insertion portion 2, the odd-shaped fitting portion 61 is engaged with the fitting hole portion 62 in an inserted state. Therefore, the fitting portion fitting the fitting hole portion 62 and the odd-shaped fitting portion 61 can restrict the rotation of the imaging unit 11 in the periaxial direction with respect to the imaging unit attachment hole portion 28. Further, in this embodiment, when the odd-shaped fitting portion 61 is inserted into the fitting hole portion 62, the distal end portion of the wedge 65 inserted from the wedge insertion hole 64 is fixed in a state that the wedge 65 is inserted into the V groove portion 63 as shown in FIG. 12. As a result, in the state that the fixing position of the imaging unit 11 in the axial direction is positioned at the attaching position where the distal end portion of the imaging unit 11 abuts on the cover glass 29, the axial movement of the imaging unit 11 can be restricted with respect to the imaging unit attachment hole portion 28.

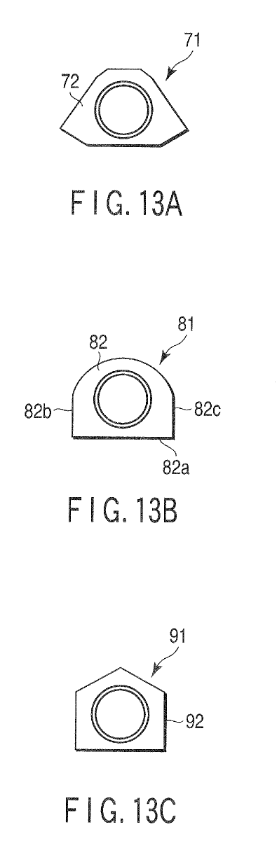

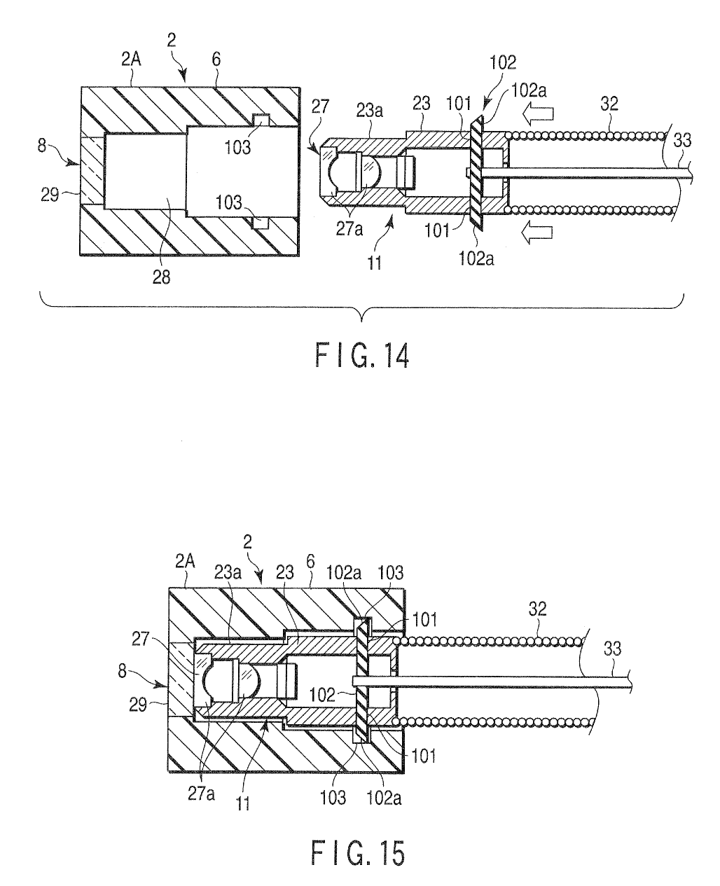

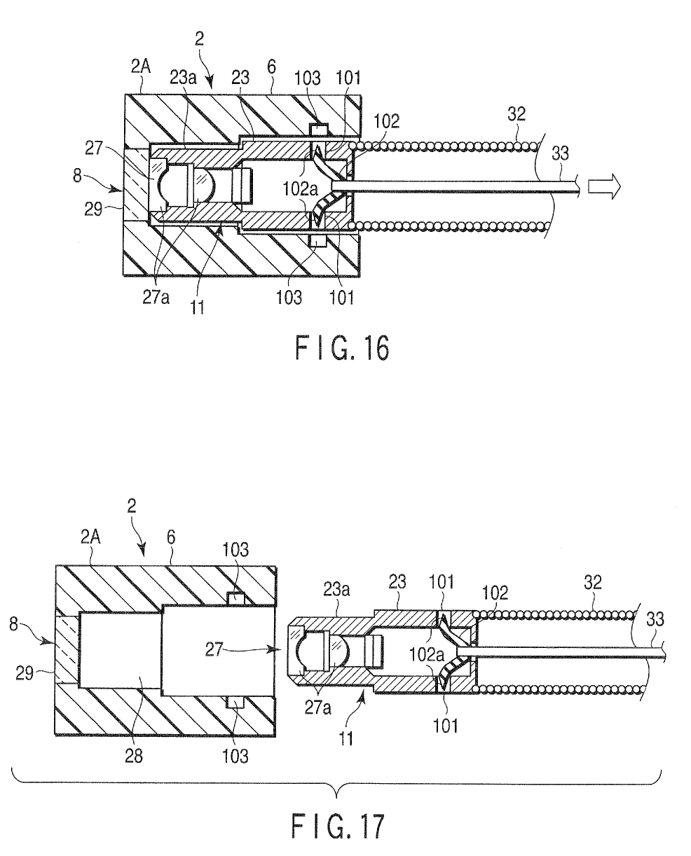

Moreover, each of FIGS. 13A to 13C shows modification of the odd-shaped fitting portion 61 of the imaging unit 11 according to a fourth embodiment (see FIGS. 11 and 12).

FIG. 13A shows a first modification of the odd-shaped fitting portion 61 of the imaging unit 11. An odd-shaped fitting portion 71 according to this modification has a substantially triangular base member 72. Additionally, each angular portion of the base member 72 is cut, and the base member is formed into a polygonal shape.

FIG. 13B shows a second modification of the odd-shaped fitting portion 61 of the imaging unit 11. An odd-shaped fitting portion 81 according to this modification has a substantially semicircular base member 82. Further, both side portions of the base member 82 are cut, and three flat surfaces 82a, 82b and 82c are formed.

FIG. 13C shows a third modification of the odd-shaped fitting portion 61 of the imaging unit 11. The odd-shaped fitting portion 91 according to this modification has a substantially pentagonal base member 92.

Furthermore, even each of the odd-shaped fitting portions 71, 81 and 91 according to the respective modifications in FIGS. 13A to 13C has an asymmetrical polygonal shape which is formed to partially protrude on the outer peripheral surface of the unit case 23 of the imaging unit 11 along the periaxial direction of the central axis of the insertion portion 2, and one flat surface is provided in the circumferential direction, thereby obtaining the same effect as the fourth embodiment.

Moreover, each of FIGS. 14 to 17 shows a fifth embodiment according to the present invention. This embodiment is obtained by changing the configuration of a lock mechanism of the imaging unit 11 with respect to the imaging unit attachment hole portion 28 of the main body 2A of the insertion portion 2 in the first embodiment (see FIGS. 1 to 6) as follows.

That is, in this embodiment, both side surfaces of the unit case 23 of the imaging unit 11 have through-holes 101 as shown in FIG. 14. A rod-shaped unit fixing member 102 formed of an elastic member such as rubber passes through each of these through-holes 101 to be moveable in the axial direction of the through-hole 101. Each locking portion 102a extended to the outer side of the through-hole 101 is provided at each of both end portions of the unit fixing member 102. A distal end portion of an operation wire 33 is fixed at the central portion of the unit fixing member 102.

Additionally, two fixing holes 103 are formed in a circumferential wall portion of an imaging unit attachment hole portion 28 of a main body 2A of an insertion portion 2. These fixing holes 103 are arranged at positions associated with the locking portions 102a at both the ends of the unit fixing member 102 of the unit case 23 in a state that a fixing position of an imaging unit 11 in the axial direction is positioned at an attaching position where a distal end portion of the imaging unit 11 abuts on a cover glass 29 when the imaging unit 11 is inserted into an imaging unit attachment hole portion 28.

Further, in this embodiment, when a closely wound coil 32 is pushed in from the outer side of an imaging unit insertion/removal portion 20, a attaching operation of attaching the imaging unit 11 to the imaging unit attachment hole portion 28 of the main body 2A of the insertion portion 2 can be carried out. At the time of this attaching operation, the locking portions 102a at both the ends of the unit fixing member 102 come into contact with the circumferential wall portion of the imaging unit attachment hole portion 28 while the imaging unit 11 is pushed in toward the front side. At this moment, the imaging unit 11 moves forward by further adding push-in force that pushes in the imaging unit 11 toward the front side, while the locking portions 102a at both the ends of the unit fixing member 102 are elastically deformed in a direction that the locking portions 102a are pushed into the through-holes 101 of the unit case 23. Further, when the imaging unit 11 is pushed in to reach the attaching position where the distal end portion of the imaging unit 11 abuts on the cover class 29, the locking portions 102a at both the ends of the unit fixing member 102 are engaged while being inserted into the two fixing holes 103 of the imaging unit attachment hole portion 28 as shown in FIG. 15. As a result, the axial position of the imaging unit 11 is positioned with respect to the imaging unit attachment hole portion 28 of the main body 2A of the insertion portion 2, thereby the imaging unit 11 being locked in a state that the axial movement is restricted.

Furthermore, when removing the imaging unit 11 from the imaging unit attachment hole portion 28 of the main body 2A of the insertion portion 2, the operation wire 33 is pulled from the outer side of the imaging unit insertion/removal portion 20. As a result, when the unit fixing member 102 bends as shown in FIG. 16, the locking portions 102a at both the ends of the unit fixing member 102 are removed from the two fixing holes 103 of the imaging unit attachment hole portion 28, thereby the imaging unit 11 being unlocked.

When the operation wire 33 is further pulled in this state, an operation of pulling and separating the imaging unit 11 from the imaging unit attachment hole portion 28 of the main body 2A of the insertion portion 2 can be carried out as shown in FIG. 17.

Therefore, in this embodiment, likewise, the operation of attaching/detaching the imaging unit 11 to/from the imaging unit attachment hole portion 28 of the main body 2A of the insertion portion 2 can be carried out by pushing/pulling the long closely wound coil 32 or the operation wire 33 from the outer side of the imaging unit insertion/removal portion 20, and hence an operator no longer needs an operation of directly grasping the small imaging unit 11. Therefore, like the first embodiment, the workability of the operation of attaching/detaching the imaging unit 11 to/from the imaging unit attachment/detachment hole portion 28 of the main body 2A of the insertion portion 2 can be improved, thus readily effecting the operation of incorporating the imaging unit 11 to the imaging unit attachment hole portion 28 of the main body 2A of the insertion portion 2 and separating the imaging unit 11 from the imaging unit attachment hole portion 28.

Other characteristic technical particulars of this invention are additionally written as follows.

### Note

(Additional Article 1) A separation-type endoscope comprising: an endoscope main body including an insertion portion and an operation portion; an imaging unit which is inserted into/removed from the main body and which is inserted into the endoscope main body in a longitudinal direction from a rear end of the insertion portion; and an flexible insertion member which is extended in the longitudinal direction of the insertion portion in the imaging unit, includes one end fixed to a distal end portion of the imaging unit, and has a length which does not change with respect to the compression in the longitudinal direction.
(Additional Article 2) The separation-type endoscope according to Additional Article 1, wherein the insertion member includes a closely wound coil.
(Additional Article 3) The separation-type endoscope according to Additional Article 1, further comprising a flexible pull-out member which is extended in the longitudinal direction of the insertion portion in the imaging unit, includes one end fixed to the distal end portion of the imaging unit, and has a length which does not change with respect to pulling in the longitudinal direction.
(Additional Article 4) The separation-type endoscope according to Additional Article 3, wherein the pull-out member includes a metal wire.
(Additional Article 5) The separation-type endoscope according to Additional Article 3, further comprising a single member (a force transmission member) configured to function as the insertion member and the pull-out member.
(Additional Article 6) The separation-type endoscope according to Additional Article 5, wherein the single member includes a resin corrugated tube.
(Additional Article 7) The separation-type endoscope according to Additional Article 5, wherein the single member includes a resin tube including a plurality of opening portions provided therein.
(Additional Article 8) The separation-type endoscope according to Additional Article 3, wherein the imaging unit includes an imaging module portion which is provided in the distal end portion of the imaging unit and to which an imaging element and its associated electronic components are attached, and a video signal cable which is extended in the longitudinal direction of the insertion unit and which includes one end connected at the distal end portion of the imaging unit.
(Additional Article 9) The separation-type endoscope according to Additional Article 8, wherein the imaging unit includes an imaging unit case which is provided at an outer periphery of the imaging module portion and with which one end of the insertion member or the pull-out member is connected.
(Additional Article 10) The separation-type endoscope according to Additional Article 9, wherein the insertion member includes a closely wound coil which includes an end portion welded and fixed to the imaging unit case.
(Additional Article 11) The separation-type endoscope according to Additional Article 9, wherein the pull-out member includes a metal wire which includes an end portion welded and fixed to the imaging unit case.
(Additional Article 12) The separation-type endoscope according to Additional Article 9, further comprising a resin single corrugated tube which includes an end portion bonded and fixed to the imaging unit case which is configured to function as the insertion member and the pull-out member.
(Additional Article 13) The separation-type endoscope according to Additional Article 9, further comprising a single resin tube which includes an opening portion provided at an outer periphery and an end portion bonded and fixed to the imaging unit case and which is configured to function as the insertion member and the pull-out member.
(Additional Article 14) The separation-type endoscope according to Additional Article 9, wherein the imaging unit case includes a fitting portion which includes an outer periphery being asymmetrical in a rotating direction, and the endoscope main body includes a main-body-side fitting portion which is provided in the distal end of the endoscope main body and which is fitted to the fitting portion of the imaging unit case.
(Additional Article 15) The separation-type endoscope according to Additional Article 14, wherein the fitting portion includes at least one flat surface provided on the outer peripheral.
(Additional Article 16) The separation-type endoscope according to Additional Article 14, wherein the fitting portion has a polygonal shape which is provided on the outer peripheral and which is asymmetrical in the rotating direction.
(Additional Article 17) The separation-type endoscope according to Additional Article 9, the imaging unit case includes an imaging unit fixing member which is provided on an outer surface of the imaging unit case and which is configured to be elastically deformed when inserting the imaging unit into the distal end portion of the endoscope main body, and the endoscope main body includes a fixing hole which is provided at the distal end portion of the endoscope main body and in which the imaging unit fixing member is fitted.
(Additional Article 18) The separation-type endoscope according to Additional Article 17, wherein the pull-out member includes a pull-out member which is connected with the imaging unit fixing member and which is configured to pull out the imaging unit fixing member from the fixing hole by a pulling operation thereof.

Additional advantages and modifications will readily occur to those skilled in the art. Therefore, the invention in its broader aspects is not limited to the specific details and representative embodiments shown and described herein. Accordingly, various modifications may be made without depart from the spirit or scope of the general inventive concept as defined by the appended claims and their equivalents.

What is claimed is:

1. An endoscope comprising:
an elongated insertion portion which is inserted into a lumen;
an operation portion which is coupled with a proximal end portion of the insertion portion;
an imaging unit which is arranged in a distal end portion of the insertion portion and which is configured to acquire an endoscopic image;
an imaging unit attachment portion which is provided at the distal end portion of the insertion portion and detachably holds the imaging unit;
an imaging unit insertion/removal portion which is provided in the operation portion and enables insertion/removal of the imaging unit with respect to the insertion portion; and
an operating mechanism which includes a distal end portion fixed to the imaging unit and a proximal end portion extended to a imaging unit insertion/removal portion side, and which is configured to perform an attachment/detachment operation of the imaging unit with respect to the imaging unit attachment portion on the imaging unit insertion/removal portion side.

2. The endoscope according to claim 1, wherein the operating mechanism includes a push-out force transmission member which is formed of a flexible elongated member extended in a central axial direction of the insertion portion and which is configured to transmit push-out force of the insertion portion in the central axial direction.

3. The endoscope according to claim 2, wherein the push-out force transmission member includes a closely wound coil.

4. The endoscope according to claim 1, wherein the operating mechanism includes a tensile force transmission member which is formed of a flexible elongated member extended in a central axial direction of the insertion portion and which is configured to transmit tensile force of the insertion portion in the central axial direction.

5. The endoscope according to claim 4, wherein the tensile force transmission member includes a metal wire.

6. The endoscope according to claim 1, wherein the operating mechanism includes:
a push-out force transmission member which is formed of a flexible elongated member extended in a central axial direction of the insertion portion and which is configured to transmit push-out force of the insertion portion in the central axial direction; and
a tensile force transmission member which is formed of a flexible elongated member extended in the central axial direction of the insertion portion and which is configured to transmit tensile force of the insertion portion in the central axial direction.

7. The endoscope according to claim 6, wherein the operating mechanism includes a single force transmission member configured to function as the push-out force transmission member and the tensile force transmission member.

8. The endoscope according to claim 7, wherein the single force transmission member includes a resin corrugated tube.

9. The endoscope according to claim 7, wherein the single force transmission member includes a perforated tube including a plurality of hole portions which are provided in a tube wall of a resin tube body and which are configured to adjust the flexibility.

10. The endoscope according to claim 1, wherein the imaging unit includes an imaging module portion to which an imaging element and its associated electronic components are attached, and a video signal cable which includes a distal end portion connected to the imaging module portion and a proximal end portion extended to an operation unit side.

11. The endoscope according to claim 10, wherein the imaging unit includes a unit case which is provided at an outer periphery of the imaging module portion and with which the operating mechanism is connected.

12. The endoscope according to claim 11, wherein the operating mechanism includes a closely wound coil as a push-out force transmission member which includes a distal end portion fixed to the unit case and which is configured to transmit push-out force of the insertion portion in a central axial direction.

13. The endoscope according to claim 11, wherein the operating mechanism includes a metal wire as a tensile force transmission member which includes a distal end portion fixed to the unit case and which is configured to transmit tensile force of the insertion unit in a central axial direction.

14. The endoscope according to claim 11, wherein the operating mechanism includes a corrugated tube which includes a distal end portion fixed to the unit case.

15. The endoscope according to claim 11, wherein the operating mechanism includes a perforated tube which includes a distal end portion fixed to the unit case.

16. The endoscope according to claim 11, wherein the unit case includes a fitting portion including an outer peripheral surface whose shape is asymmetrical in a periaxial direction of a central axis of the insertion portion, and
the imaging unit attachment portion includes a fitting hole portion which is fitted to the fitting portion of the unit case.

17. The endoscope according to claim 16, wherein the fitting portion includes at least one flat surface provided on the outer peripheral surface.

18. The endoscope according to claim 16, wherein the fitting portion has a polygonal shape which is provided on the outer peripheral surface and which is asymmetrical in the periaxial direction of the central axis of the insertion portion.

19. The endoscope according to claim 11, wherein the unit case includes a unit fixing member which is provided on an outer surface of the unit case and which is configured to be elastically deformed at the time of an operation of attaching the imaging unit to the imaging unit attachment portion, and
the imaging unit attachment portion includes a fixing hole in which the unit fixing member is fitted.

20. The endoscope according to claim 19, wherein the operating mechanism includes a tensile force transmission member which is connected with the unit fixing member and which is configured to transmit tensile force of the insertion portion in a central axial direction and pull out the unit fixing member from the fixing hole by a pulling operation thereof.
